(19) United States
(12) Patent Application Publication
(10) Pub. No.: US 2020/0268547 A1
(43) Pub. Date: Aug. 27, 2020

(54) Cooling System

(71) Applicant: OxiCool Inc., Malvern, PA (US)

(72) Inventor: Ravikant T. Barot, Phoenixville, PA (US)

(73) Assignee: OxiCool Inc., Malvern, PA (US)

(21) Appl. No.: 16/283,146

(22) Filed: Feb. 22, 2019

(51) Int. Cl. A61F 7/00; A61G 1/04

(52) U.S. Cl. CPC A61F 7/0053; A61F 2007/0058; A61G 1/04

(57) ABSTRACT

Technologies are described herein for cooling systems. In some aspects, a cooling system includes an adsorbent chamber and an evaporator. The cooling effect caused by the evaporation of refrigerant in the evaporator is applied to a patient on a support device, such as a gurney or stretcher, cooling the patient. The evaporated refrigerant is adsorbed by absorbent in the adsorbent chamber.

### BACKGROUND

During some traumatic events, a patient undergoing treatment may need to be cooled rapidly. For example, there is a commonly recognized “golden hour” for heatstroke treatment. The golden hour is the time period after the onset of heat stroke in which therapy can be extremely effective. During this time, if the patient is removed from the heat source and is cooled, the potential effects of the heatstroke diminish greatly. In another example, it has recently been found that a patient's body temperature after a cardiac arrest can significantly influence the patient's chances of recovery.

Conventional means of rapidly cooling often involve large compression-based refrigeration units, wet towels, large bags of ice, and the like. While effective in some instances, the transportation, use, and re-use of these conventional means can often be difficult. For example, towels are bulky and need copious amounts of water to be effective. It is expensive, and often, impractical to carry ice around in the off-chance that it would need to be used. Compression-based refrigeration units require an energy source and, due to the weight of the compressor, can often be heavy.

It is with respect to these and other considerations that the disclosure made herein is presented.

### SUMMARY

Technologies are described herein for a cooling device that uses the crystalline structure of an adsorbent in a cooling device and system. In some examples, the cooling device uses an adsorbent chamber and an evaporator integrated into a medical device. In some examples, the medical device is a stretcher or gurney. The cooling device reduces the temperature of either surfaces or the air surrounding the patient. In some examples, the cooling device is designed to be transportable or have a weight and size that allows the proper functioning and use of the stretcher with the cooling device.

During use, the adsorbent in the adsorbent chamber adsorbs water vapor generated in the evaporator. The evaporation of the water reduces the temperature of the water in the evaporator. Through the action of heat conduction, the temperature in one or more portions of the support structure are reduced. When a patient is lain upon the support structure, the cooled portion(s) of the support structure act to cool the patient.

In some examples, the cooling device uses a pre-charged absorbent chamber and a pre-charged evaporator. A pre-charged absorbent chamber is a chamber having the adsorbent in at least a partial vacuum. A pre-charged evaporator is an evaporator having a coolant (such as water) at or near atmospheric pressure. The adsorbent chamber and the evaporator can either be connected prior to use, or connected at the time of use.

In some examples, the cooling device includes an evaporator fluidly coupled to an adsorbent chamber. In a cooling mode, the refrigerant vaporizes, causing the evaporator to absorb heat. The adsorbent chamber receives the refrigerant vapor. The adsorbent chamber includes an adsorbent. The adsorbent adsorbs the refrigerant vapor in the crystalline structure of the adsorbent. In a recharging mode, heat is applied to the adsorbent, causing desorption of the refrigerant from the adsorbent. In some examples, more than one evaporator and/or adsorbent chamber can be used to maintain at least a portion of the cooling device in a cooling mode while allowing a recharging mode.

This Summary is provided to introduce a selection of technologies in a simplified form that are further described below in the Detailed Description. This Summary is not intended to identify key features or essential features of the claimed subject matter, nor is it intended that this Summary be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1 is an illustration showing the use of a cooling system.

FIG. 2 is an illustration of a cooling system.

FIG. 3 is an illustration of a cooling system using a detachable adsorbent chamber and a cooling controller.

FIG. 4 is an illustration showing a support structure with cooling pads.

FIG. 5 is a flow chart illustrating a method for operating a cooling system.

FIG. 6 is a computer architecture diagram illustrating an illustrative computer hardware and software architecture for a computing system capable of implementing aspects presented herein.

### DETAILED DESCRIPTION

The following detailed description is directed to various examples of a cooling system that uses the crystalline structure of an adsorbent to provide a cooling effect. For example, the adsorbent can be zeolite. It should be understood that, while various examples described herein are described in terms of the use of zeolite, the presently disclosed subject matter is not necessary limited to zeolite, as other suitably equipped adsorbents, including, but not limited to, molecular sieves, metal organic frameworks, and electrically activated adsorbents, may be used. In some examples, electrically activated adsorbents, such as activated charcoal, can be adsorbents configured with an electrical charge to adsorb molecules.

In the following detailed description, references are made to the accompanying drawings that form a part hereof, and in which are shown by way of illustration specific examples. Referring now to the drawings, aspects of technologies for cooling devices will be presented.

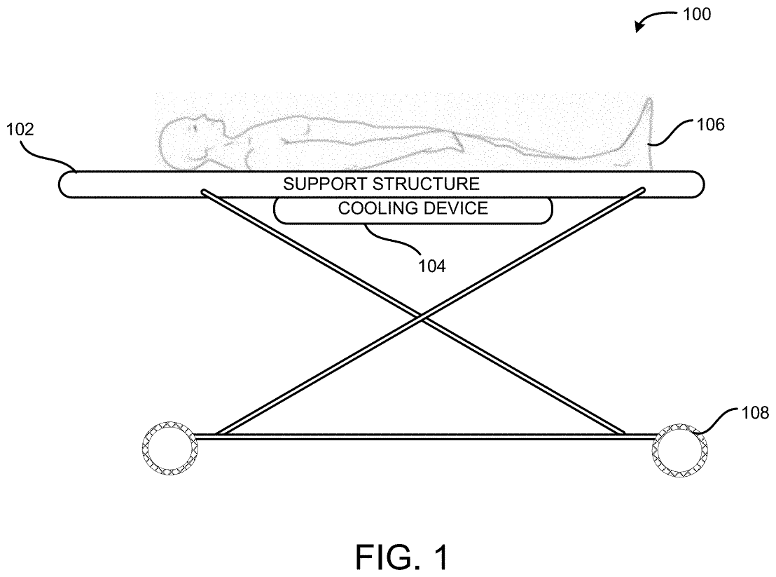

FIG. 1 is an illustration of a cooling device system 100. In some examples, the cooling device system 100 includes a support structure 102 and a cooling device 104. In some examples, the support structure 102 can be a gurney or stretcher used to transport a patient 106. The support structure 102 can include wheels 108 or can be moved using other means, such as being held aloft with human effort. The presently disclosed subject matter is not limited to any particular means of transport or example of the support structure 102.

In some examples, it may be desirable or necessary to decrease the temperature of the patient 106. For example, it has been shown that reducing the internal temperature of a person that has suffered from cardiac arrest can reduce or eliminate the chances of the patient suffering from brain damage caused by the cardiac arrest. In tests, it has been shown that lowering the brain temperature can protect brain cells by decreasing their energy requirement, decreasing inflammation, and reducing the chances of the introduction of toxins.

Another instance in which the reduction of body temperature may increase the chance of survival is after suffering a traumatic brain injury. For example, bicycle and motorcycle riders (as well as motorists) can suffer a traumatic brain injury in an accident. Another example are stroke victims. Reducing the temperature of the brain (as well as the body core temperature in other instances) can protect brain cells in the same manner described above. The presently disclosed subject matter is not limited to any particular reason for cooling a patient. Further, the presently disclosed subject matter is not limited to use on a particular body part, as various aspects of the presently disclosed subject matter can be used to cool various body parts.

The support structure 102 in conjunction with the cooling device 104 provide cooling capabilities to the patient 106. The patient 106 may have suffered from a traumatic injury in which cooling a part of the body of the patient 106 may decrease injury or increase the likelihood of recovery. An attendee of the patient 106, such as a first responder, can activate the cooling device 104 to provide cooling. In some examples, the attendee can place the cooling device 104 in an appropriate location to focus cooling on a particular portion of the body of the patient 106. These and other aspects of the cooling system 100 are described in more detail below.

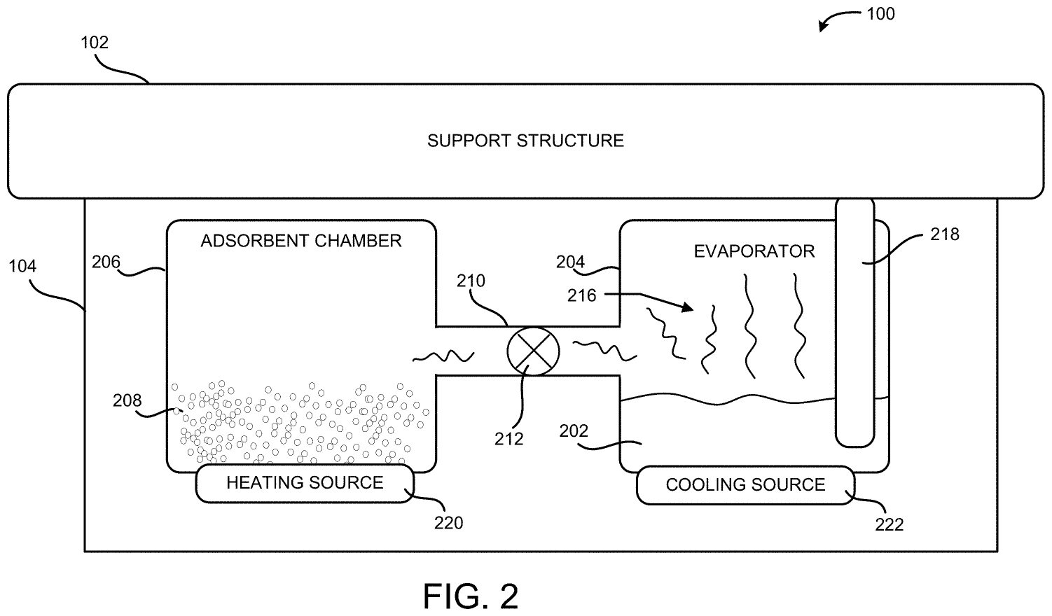

FIG. 2 is a diagram showing an example of the cooling device 104 for use in the cooling system 100. In some examples, the cooling device 104 is a closed-system, whereby a volume of working fluid (refrigerant) is maintained within the cooling device 104. The cooling device 104 includes a refrigerant 202 contained within an evaporator 204. In some examples, the refrigerant 202 is water. In some examples, the refrigerant 202 is pure water. In some examples, the refrigerant 202 is substantially pure water. In some examples, the refrigerant 202 is water containing no additives. In other systems, water containing adjuvants may be desired as the refrigerant 202. An example of useful adjuvants is an anti-microbial (e.g., bactericidal or fungicidal) composition. In some examples, the refrigerant 202 does not contain materials which would interfere with operation of the cooling device 104 in its operation. Thus, in some examples, glycols and other antifreeze agents can be excluded from the refrigerant 202, at least in amounts effective for storing cooling device 104 in ambient conditions around or below the freezing point of the refrigerant 202.

In some examples, evaporator 204 is fluidly coupled to an adsorbent chamber 206 containing an adsorbent 208. In some examples, adsorbent 208 is a material configured to adsorb and desorb the refrigerant 202. In some examples, the adsorbent 208 is configured to provide adsorption of vaporized refrigerant 216 from the evaporator 204 in a cooling mode and configured to provide desorption of the refrigerant 202 back into the evaporator 204 in a recharging mode.

In some examples, the adsorbent 208 exhibits a high ability to adsorb refrigerant 202 and to remain in an adsorbed state over practical lengths of time, while maintaining physical and physicochemical form and function. Such materials may be useful when they exhibit a high ability to adsorb water, efficient and effectively reversible desorption of water upon application of heat energy, and physical and physicochemical stability during and following repeated adsorption and desorption cycles.

In some examples, the adsorbent 208 includes a desiccant material. In some examples, the adsorbent 208 is a desiccant. In some examples, the adsorbent 208 is zeolite. A zeolite may be described as, but without limitation, hydrous aluminum silicate in porous granules. Exemplary zeolites that may be used include analxime, chabazite, heulandite, natrolite, phillipsite and stilbite. In some examples, the adsorbent 208 is any drying agent that maintains its physical structure when substantially fully contacted with water. In other examples, the adsorbent 208 is any adsorptive and/or absorptive material including but not limited to diatomaceous earth, activated alumina, silica gel, calcium aluminosilicate clay, molecular sieves (e.g., electrically charged molecular sieves), metal organic framework materials, activated carbon, and/or lithium chloride. In other examples, the adsorbent 208 may be an electrically chargeable and dischargeable material (e.g., a porous slab or particles of material such as a metal including aluminum, stainless steel and alloys thereof) such that electrical energy is used to control the electrical charge of the pores of the material to adsorb and desorb the refrigerant 202 from the adsorbent 208.

The evaporator 204 is fluidly coupled to the adsorbent chamber 206 via a fluid passageway 210 such as a pipe or conduit. In one example, the fluid passageway 210 includes a valve 212 that controls the fluid coupling between the evaporator 204 and the adsorbent chamber 206. In some examples, the refrigerant 202 is hermetically sealed within the cooling device 104.

In some examples, the adsorbent chamber 206 is fluidically coupled and isolated from the evaporator 204 by passageway 210 and valve 212. In some examples, in a pre-charged mode, the adsorbent chamber 206 is isolated from the evaporator 204 and is at a partial or full vacuum. However, it should be understood that, in some examples, the presently disclosed subject matter does not require the use of a partial or full vacuum. In some examples, the adsorption of the vaporized refrigerant 216 causes a reduction in pressure at the refrigerant 202 in the evaporator 204, causing evaporation of the refrigerant 202 and, thus, reducing the temperature of the refrigerant 202.

In some examples, in the pre-charged mode, the evaporator 204 is at or near atmospheric pressure. At the time of use, the valve 212 is opened. In some examples, the valve 212 can be a thermally actuated valve that opens the passageway 210 between the evaporator 204 and the adsorbent chamber 206 when a calibrated temperature is reached. In some examples, the valve 212 may be actuated using a bi-metallic coil, plate, diaphragm, as well as an impregnated wax element and other temperature reacting technologies.

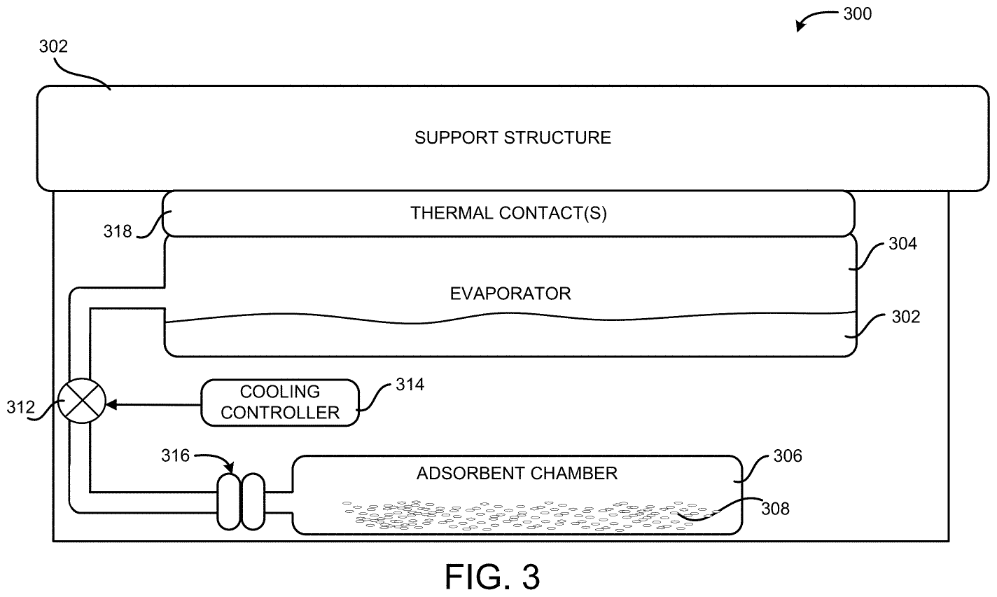

In further examples, the valve 212 can be a foil or another barrier that, when breached, allows the adsorbent chamber 206 to be fluidically coupled to the evaporator 204. For example, the evaporator 204 and the adsorbent chamber 206 may be separable or detachable components. The adsorbent chamber 206 may be connectable to the evaporator 204 using a quick connect mechanism (shown by way of example in FIG. 3). The adsorbent chamber 206 can include a pierce-able physical barrier (such as a metal foil) that maintains the vacuum of the adsorbent chamber 206 when not pierced. When connected, a piercing mechanism (not shown) installed in the passageway 210 can pierce the physical barrier, completing the fluidic connection between the evaporator 204 and the adsorbent chamber 206. The presently disclosed subject matter, however, is not limited to any particular technology for connecting the evaporator 204 to the adsorbent chamber 206.

The vacuum in the adsorbent chamber 206 helps to reduce the pressure in the evaporator 204. At a particular pressure (or partial pressure), the refrigerant 202 in the evaporator 204 begins to boil (or evaporate) due to the reduced pressure in the evaporator 204. The particular pressure at which boiling occurs depends on, among other things, the temperature of the refrigerant 202. As the refrigerant 202 boils, creating the vaporized refrigerant 216, the temperature of the refrigerant 202 decreases due to the latent heat of evaporation. The vaporized refrigerant 216 moves through the passageway 210 and is adsorbed by the adsorbent 208. As the vaporized refrigerant 216 leaves the evaporator 204, the pressure in the evaporator 204 may continue to decrease. The adsorbent 208 can continue to adsorb the vaporized refrigerant 216 until the adsorbent 208 is fully or at least partially saturated. It should be noted that the presently disclosed subject matter does not require boiling to occur. In some examples, evaporation can occur due to the partial pressure (saturation pressure) of the water vapor above the refrigerant 202 caused by the adsorbent 208.

As noted above, as the refrigerant 202 is vaporized, the temperature of the refrigerant 202 is reduced (due to the latent heat of vaporization). The reduction in temperature of the refrigerant 202 can be used to cool, or more accurately, remove heat from, the patient 106. In some examples, a heat transfer device 218 may be used to transfer heat from the patient 106. The heat transfer device 218 may vary. For example, the heat transfer device 218 can be a heat exchanger through which fluid is moved through the heat transfer device 218. Fluid near the patient 106 can take in heat from the patient 106.

The heat from the warmed fluid in the heat transfer device 218 can be transferred into the refrigerant 202. In some examples, the heat transfer device 218 can use air, water, or other fluid. In some examples, a surface of the evaporator 204 can be placed in a position proximate to the patient 106. As the temperature of the refrigerant 202 is reduced, the temperature of the surface of the evaporator 204 is reduced, providing a cooling surface to the patient 106 when placed proximate to the patient 106. It should be noted, however, that the presently disclosed subject matter is not limited to any particular heat transfer mechanism.

After use, the adsorbent 208 in the adsorbent chamber 206 can be partially or fully saturated, reducing the ability of the adsorbent 208 to adsorb additional refrigerant 202. In this instance, the cooling device 104 can be reset or placed back into a pre-charge mode by applying a heating source 220 to the adsorbent 208. The heating of the adsorbent causes the adsorbent 208 to desorb the refrigerant. Using a cooling source 222 in the evaporator 204, the desorbed refrigerant (currently in a vapor stage) can be condensed into liquid in the evaporator 204. The presently disclosed subject matter is not limited to any particular heating source 220 or cooling source 222.

FIG. 3 is an illustration of a cooling system 300 using a heat transfer device. In FIG. 3, the cooling system 300 includes an evaporator 304 and an adsorbent chamber 306. A refrigerant 302 evaporates in the evaporator 304 and is adsorbed by an adsorbent 308 in the adsorbent chamber 306. As discussed above, the adsorbent chamber 306 can be at a vacuum in a pre-charged (or pre-use) mode, whereby a valve separates the interior of the evaporator 304 from the interior of the adsorbent chamber 306. To use the cooling system 300, a cooling controller 314 causes the valve 312 to open. In some examples, the cooling controller 314 can receive an input to initiate the cooling system 300.

As noted above, in some examples, the adsorbent chamber 306 may be a separable unit from the cooling system 300. For example, the adsorbent chamber 306 can be a portable or hand-held component that can be connected to the evaporator 304. In some examples, the adsorbent chamber 306 can be interchangeable with other adsorbent chambers (not shown). In FIG. 3, the adsorbent chamber 306 can be mechanically connected to the evaporator 304. In some examples, once mechanically connected, the interior of the adsorbent chamber 306 is fluidically connected to the interior of the evaporator 304.

The cooling system 300 also includes quick disconnect mechanism 316. In some examples, the quick disconnect mechanism 316 is a quick acting coupling comprising a plug and socket that fluidically couples and decouples the adsorbent chamber 306 from the evaporator 304. Some examples of the quick disconnect mechanism include, but are not limited to, a ball-lock coupling, a roller-lock coupling, a pin-lock coupling, a ring-lock coupling, and a cam-lock coupling.

To remove heat from a patient, the cooling system 300 can include a thermal contact 318. The thermal contact 318 can be a heat transfer interface between the evaporator 304 and an area of the support structure. As noted by way of example above, as the refrigerant 302 cools from the evaporation of the refrigerant 302, the temperature of one or more surfaces of the evaporator 304 may decrease. Heat from a patient may be transferred through the thermal contact 318 into the evaporator 304.

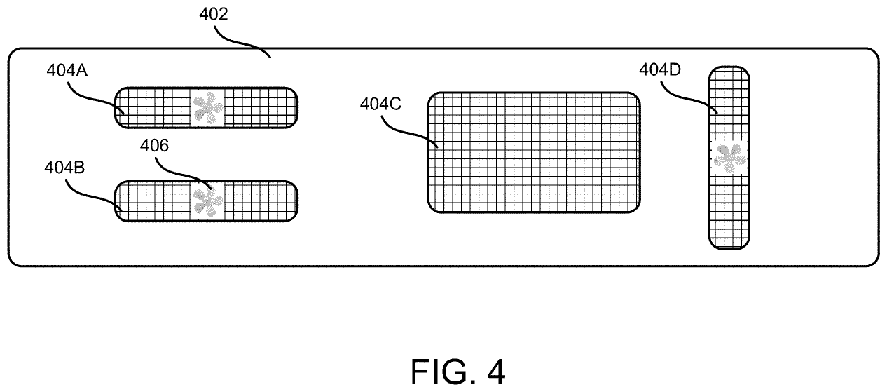

FIG. 4 is an example of a support structure 402 that may be used in conjunction with various examples of a cooling system. The support structure 402 includes cooling pads 404A-404D. The cooling pads 404A-404D are areas of the support structure 402 that transfer heat from a patient into a cooling system, such as the cooling systems described in FIGS. 1-3.

The cooling pads 404A-404D can be used by medical personnel to cool at portion of a patient. For example, the cooling pad 404A can be used to cool a right leg of a patient, the cooling pad 404B can be used to cool a left leg of a patient, the cooling pad 402C can be used to cool the torso of a patient, and the cooling pad 404D can be used to cool the head/neck of a patient. In some examples, additional cooling may be provided by a fan 406 or other means. For example, the fan 406 may help draw cooler air around the patient.

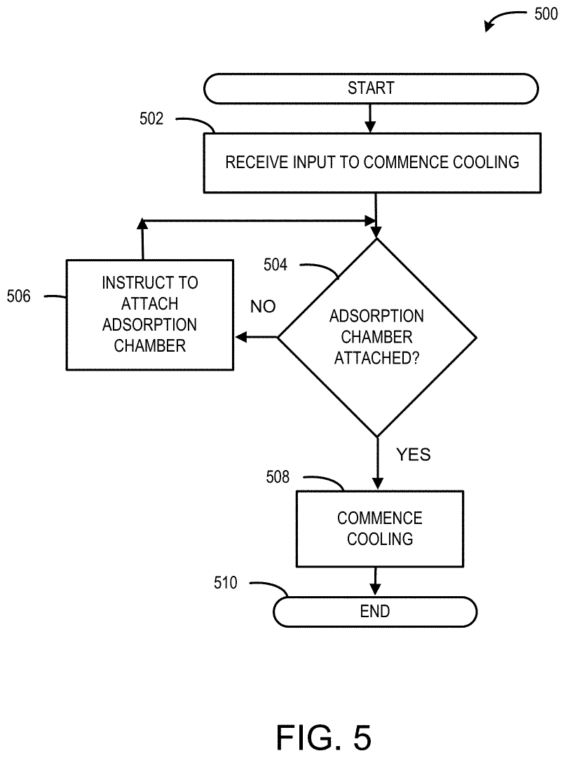

Turning now to FIG. 5, aspects of a method 500 for operating a cooling system, such as the cooling system 200, will be described in detail. It should be understood that the operations of the method 500 are not necessarily presented in any particular order and that performance of some or all of the operations in an alternative order(s) is possible and is contemplated. The operations have been presented in the demonstrated order for ease of description and illustration. Operations may be added, omitted, and/or performed simultaneously, without departing from the scope of the appended claims.

It also should be understood that the illustrated method 500 can be ended at any time and need not be performed in its entirety. Some or all operations of the method 500, and/or substantially equivalent operations, can be performed by execution of computer-readable instructions included on a computer-storage media, as defined herein. The term “computer-readable instructions,” and variants thereof, as used in the description and claims, is used expansively herein to include routines, applications, application modules, program modules, programs, components, data structures, algorithms, and the like. Computer-readable instructions can be implemented on various system configurations, including single-processor or multiprocessor systems, electronic control units, electronic control modules, programmable logic controllers, minicomputers, mainframe computers, personal computers, hand-held computing devices, microprocessor-based, programmable consumer electronics, combinations thereof, and the like. In some examples, instructions can be provided by a logic hard wired or hard encoded control system using relays, transistors, mosfets, logic gates, and the like. Computer-storage media does not include transitory media.

Thus, it should be appreciated that the logical operations described herein can be implemented as a sequence of computer implemented acts or program modules running on a computing system, and/or as interconnected machine logic circuits or circuit modules within the computing system. The implementation is a matter of choice dependent on the performance and other requirements of the computing system. Accordingly, the logical operations described herein are referred to variously as states, operations, structural devices, acts, or modules. These operations, structural devices, acts, and modules may be implemented in software, in firmware, in special purpose digital logic, and any combination thereof. For purposes of illustrating and describing the technologies of the present disclosure, the method 500 disclosed herein is described as being performed by appropriate components of the cooling system 300 via execution of computer executable instructions. As such, it should be understood that the described configuration is illustrative, and should not be construed as being limiting in any way.

The method 500 begins at operation 502, where an input is received to commence cooling. For example, the input can be generated by a first responder or another medical professional that determines that cooling may be required. In other examples, the input can be generated when a condition occurs. For example, the input can be received from an accelerometer, indicating that an accident has occurred. In another example, the input can be from a thermometer, indicating that a particular temperature has been reached (such as an internal temperature of a patient). The presently disclosed subject matter is not limited to any particular input.

The method 500 continues to operation 504, where a determination is made as to whether or not an adsorbent chamber is attached. As noted above, in some examples, the cooling system may use detachable adsorbent chambers. In some examples, it may be desirable or necessary to determine if an adsorbent chamber is attached, especially in emergency conditions where a lot of activity is occurring.

The method 500 continues to operation 506, where if the determination is made that an adsorbent chamber is not attached, an instruction is provided to attach an adsorbent chamber. The instruction can be, among various possibilities, a sound, a light, text, or other means to inform a user that an adsorbent chamber is not attached.

If at operation 504 it is determined that the adsorbent chamber is attached, the method continues to operation 508, where cooling is commenced. As noted above, cooling may be commenced automatically upon a fluidic connection being made between an adsorbent chamber and an evaporator or upon the opening of a valve to fluidically connect the adsorbent chamber to the evaporator. The presently disclosed subject matter is not limited to any particular manner of connection. The method 500 thereafter ends at operation 510.

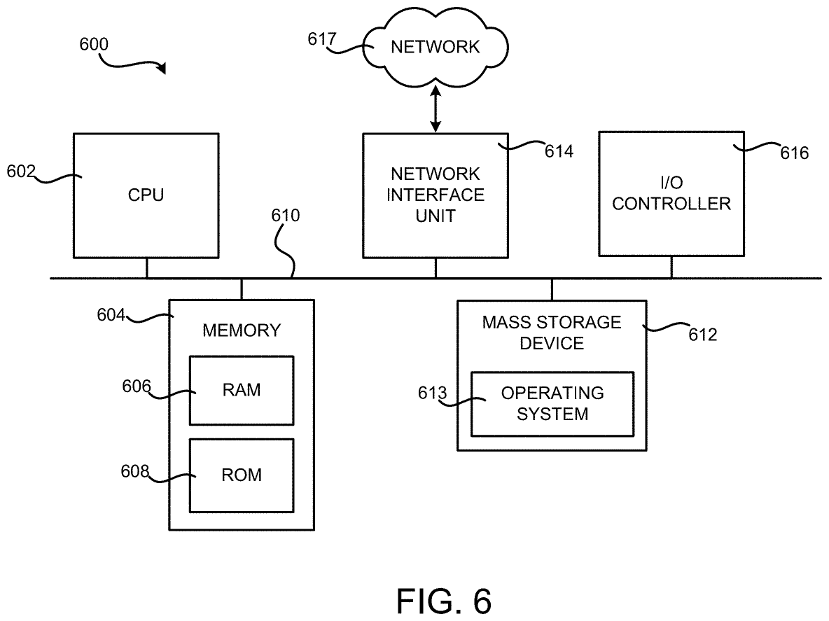

FIG. 6 illustrates an illustrative computer architecture 600 for a device capable of executing the software components described herein for operating a cooling system, including the cooling controller 314 of FIG. 3. Thus, the computer architecture 600 illustrated in FIG. 6 illustrates an architecture for a server computer, mobile phone, a smart phone, a desktop computer, a netbook computer, a tablet computer, and/or a laptop computer. The computer architecture 600 may be utilized to execute any aspects of the software components presented herein.

The computer architecture 600 illustrated in FIG. 6 includes a central processing unit 602 (“CPU”), a system memory 604, including a random access memory 606 (“RAM”) and a read-only memory (“ROM”) 608, and a system bus 610 that couples the memory 604 to the CPU 602. A basic input/output system containing the basic routines that help to transfer information between elements within the computer architecture 600, such as during startup, is stored in the ROM 608. The computer architecture 600 further includes a mass storage device 612 for storing an operating system 613 and one or more application programs for operating a cooling system.

The mass storage device 612 is connected to the CPU 602 through a mass storage controller (not shown) connected to the bus 610. The mass storage device 612 and its associated computer-readable media provide non-volatile storage for the computer architecture 600. Although the description of computer-readable media contained herein refers to a mass storage device, such as a hard disk or CD-ROM drive, it should be appreciated by those skilled in the art that computer-readable media can be any available computer storage media or communication media that can be accessed by the computer architecture 600.

Communication media includes computer readable instructions, data structures, program modules, or other data in a modulated data signal such as a carrier wave or other transport mechanism and includes any delivery media. The term “modulated data signal” means a signal that has one or more of its characteristics changed or set in a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

By way of example, and not limitation, computer storage media may include volatile and non-volatile, removable and non-removable media implemented in any method or technology for storage of information such as computer-readable instructions, data structures, program modules or other data. For example, computer storage media includes, but is not limited to, RAM, ROM, EPROM, EEPROM, flash memory or other solid state memory technology, CD-ROM, digital versatile disks (“DVD”), HD-DVD, BLU-RAY, or other optical storage, magnetic cassettes, magnetic tape, magnetic disk storage or other magnetic storage devices, or any other medium which can be used to store the desired information and which can be accessed by the computer architecture 600. For purposes the claims, a “computer storage medium” or “computer-readable storage medium,” and variations thereof, do not include waves, signals, and/or other transitory and/or intangible communication media, per se. For the purposes of the claims, “computer-readable storage medium,” and variations thereof, refers to one or more types of articles of manufacture.

According to various configurations, the computer architecture 600 may operate in a networked environment using logical connections to remote computers through a network such as the network 617. The computer architecture 600 may connect to the network 617 through a network interface unit 614 connected to the bus 610. It should be appreciated that the network interface unit 614 also may be utilized to connect to other types of networks and remote computer systems. The computer architecture 600 also may include an input/output controller 616 for receiving and processing input from a number of other devices, including a keyboard, mouse, or electronic stylus (not shown in FIG. 6). Similarly, the input/output controller 616 may provide output to a display screen, a printer, or other type of output device (also not shown in FIG. 6).

It should be appreciated that the software components described herein may, when loaded into the CPU 602 and executed, transform the CPU 602 and the overall computer architecture 600 from a general-purpose computing system into a special-purpose computing system customized to facilitate the functionality presented herein. The CPU 602 may be constructed from any number of transistors or other discrete circuit elements, which may individually or collectively assume any number of states. More specifically, the CPU 602 may operate as a finite-state machine, in response to executable instructions contained within the software modules disclosed herein. These computer-executable instructions may transform the CPU 602 by specifying how the CPU 602 transitions between states, thereby transforming the transistors or other discrete hardware elements constituting the CPU 602.

Encoding the software modules presented herein also may transform the physical structure of the computer-readable media presented herein. The specific transformation of physical structure may depend on various factors, in different implementations of this description. Examples of such factors may include, but are not limited to, the technology used to implement the computer-readable media, whether the computer-readable media is characterized as primary or secondary storage, and the like. For example, if the computer-readable media is implemented as semiconductor-based memory, the software disclosed herein may be encoded on the computer-readable media by transforming the physical state of the semiconductor memory. For example, the software may transform the state of transistors, capacitors, or other discrete circuit elements constituting the semiconductor memory. The software also may transform the physical state of such components in order to store data thereupon.

As another example, the computer-readable media disclosed herein may be implemented using magnetic or optical technology. In such implementations, the software presented herein may transform the physical state of magnetic or optical media, when the software is encoded therein. These transformations may include altering the magnetic characteristics of particular locations within given magnetic media. These transformations also may include altering the physical features or characteristics of particular locations within given optical media, to change the optical characteristics of those locations. Other transformations of physical media are possible without departing from the scope and spirit of the present description, with the foregoing examples provided only to facilitate this discussion.

In light of the above, it should be appreciated that many types of physical transformations take place in the computer architecture 600 in order to store and execute the software components presented herein. It also should be appreciated that the computer architecture 600 may include other types of computing devices, including hand-held computers, embedded computer systems, personal digital assistants, and other types of computing devices known to those skilled in the art. It is also contemplated that the computer architecture 600 may not include all of the components shown in FIG. 6, may include other components that are not explicitly shown in FIG. 6, or may utilize an architecture completely different than that shown in FIG. 6.

Various aspect of the presently disclosed subject matter may be considered in view of the following clauses:

Clause 1: [to be completed when claims finalized]

Based on the foregoing, it should be appreciated that technologies for a cooling system have been disclosed herein. Although the subject matter presented herein has been described in language specific to structural features, methodological and transformative acts, and specific machinery, it is to be understood that the invention defined in the appended claims is not necessarily limited to the specific features, acts, or media described herein. Rather, the specific features, acts and mediums are disclosed as example forms of implementing the claims.

The subject matter described above is provided by way of illustration only and should not be construed as limiting. Various modifications and changes may be made to the subject matter described herein without following the example configurations and applications illustrated and described, and without departing from the true spirit and scope of the present invention, aspects of which are set forth in the following claims.

What is claimed is:

1. A cooling system, comprising:
a support structure designed to support a patient;
a cooling device comprising
an evaporator containing refrigerant, and
an adsorbent chamber fluidically coupleable to the evaporator, the adsorbent chamber containing an adsorbent that adsorbs the refrigerant in a cooling mode, causing the temperature of the refrigerant to decrease; and
a heat transfer device that transfers heat from a least a portion of the patient to the evaporator to provide a cooling effect to the patient.

2. The cooling system of claim 1, further comprising a valve to fluidically isolate the evaporator from the adsorbent chamber in a pre-charge mode and fluidically couple the evaporator to the adsorbent chamber in a cooling mode.

3. The cooling system of claim 1, wherein the support device comprises a stretcher or gurney.

4. The cooling system of claim 1, further comprising a cooling controller that causes the cooling system to enter the cooling mode from a pre-charge mode.

5. The cooling system of claim 1, wherein the cooling controller operates a valve to fluidically cause the cooling system to enter the cooling mode.

6. The cooling system of claim 1, wherein the adsorbent comprises zeolite.

7. The cooling system of claim 1, wherein the adsorbent comprises a metal organic framework or an electrically activated adsorbent.

8. The cooling system of claim 1, wherein the refrigerant comprises water.

9. The cooling system of claim 1, wherein the heat transfer device comprises a cooling pad located proximate to a part of the patient to be cooled.

10. The cooling system of claim 9, wherein the cooling pad further comprises a fan to provide additional cooling.

11. The cooling system of claim 1, wherein the adsorbent chamber is at a partial vacuum in a pre-charge mode.

12. The cooling system of claim 1, wherein the adsorbent chamber comprises a metal foil to maintain a partial vacuum in the adsorbent chamber when not fluidically coupled to the evaporator.

13. The cooling system of claim 1, further comprising a quick connect mechanism to fluidically couple and decouple the adsorbent chamber to the evaporator.

14. A method of operating a cooling system, the method comprising:
providing a support structure designed to support a patient;
providing an evaporator containing refrigerant;
providing an adsorbent chamber fluidically coupleable to the evaporator, the adsorbent chamber containing an adsorbent that adsorbs the refrigerant in a cooling mode;
providing a heat transfer device that transfers heat from a least a portion of the patient to the evaporator to provide a cooling effect to the patient; and
initiating cooling of a portion of the patient on the support structure by fluidically coupling the adsorbent chamber to the evaporator to provide for the adsorption of the refrigerant in the adsorbent, causing the temperature of the refrigerant to decrease.

15. The method of claim 14, further comprising operating a valve to fluidically decouple the evaporator from the adsorbent chamber.

16. The method of claim 14, wherein fluidically coupling the adsorbent chamber to the evaporator comprises opening a valve in a passageway between the adsorbent chamber and the evaporator.

17. The cooling system of claim 1, wherein the support device comprises a stretcher or gurney.

18. The cooling system of claim 1, wherein the adsorbent comprises zeolite, a metal organic framework or an electrically activated adsorbent.

19. The cooling system of claim 1, wherein the refrigerant comprises water.

20. A cooling system, comprising:
a support structure designed to support a patient;
a cooling device comprising
an evaporator containing refrigerant, and
an adsorbent chamber fluidically coupleable to the evaporator, the adsorbent chamber containing zeolite that adsorbs the refrigerant in a cooling mode, causing the temperature of the refrigerant to decrease, wherein the adsorbent chamber is detachably connected to the cooling system; and
a heat transfer device that transfers heat from a least a portion of the patient to the evaporator to provide a cooling effect to the patient.
